(19) United States
(12) Patent Application Publication
(10) Pub. No.: US 2024/0165024 A1
(43) Pub. Date: May 23, 2024

(54) MYCOPHENOLATE ORAL SUSPENSION

(71) Applicant: LIQMEDS WORLDWIDE LIMITED, Hayes (GB)

(72) Inventors: Sandip MEHTA, Ahmedabad (IN); Manish UMRETHIA, Ahmedabad (IN); Henil PATEL, Ahmedabad (IN); Jayanta Kumar MANDAL, Ahmedabad (IN)

(21) Appl. No.: 18/422,105

(22) Filed: Jan. 25, 2024

Related U.S. Application Data
(63) Continuation of application No. 16/494,030, filed on Sep. 13, 2019, now Pat. No. 11,918,684.

(30) Foreign Application Priority Data
Mar. 13, 2017 (IN) 201721008648

(51) Int. Cl. A61K 9/00; A61K 9/10; A61K 47/02; A61K 47/10; A61K 47/14; A61K 47/26; A61K 47/36

(52) U.S. Cl. CPC A61K 9/0095; A61K 9/0053; A61K 9/10; A61K 47/02; A61K 47/10; A61K 47/14; A61K 47/26; A61K 47/36

(57) ABSTRACT

Disclosed herein is a pharmaceutical oral suspension, comprising: mycophenolate mofetil in an amount of about 200 mg/mL; one or more cosolvents comprising ethanol, a polyethylene glycol, a sorbitol, glycerin, propylene glycol, benzyl alcohol, or a combination thereof in an amount of from about 5% w/w to about 30% w/w; one or more pharmaceutically acceptable excipients selected from a suspending agent, a preservative, a sweetener, an antifoaming agent, a wetting agent, a buffering agent, and a flavoring agent; and a vehicle comprising water. The pharmaceutical oral suspension has improved stability, palatability with high dose of active ingredient.

### FIELD OF THE INVENTION

The present invention relates to the oral liquid pharmaceutical composition of immunosuppressive agents. More particularly, the present invention relates to oral suspension composition comprises of immunosuppressive agent with improved stability and palatability with high dose of active ingredient.

### BACKGROUND OF THE INVENTION

Immunosuppressive are medications that help to suppress the immune system. Typically they are used in patients who received organ transplants to help and prevent their bodies from rejecting the transplanted organ. Also immunosuppressant drugs are used to treat autoimmune diseases. With an autoimmune disease, the immune system attacks the body's own tissue. Because immunosuppressant drugs weaken the immune system, they suppress this reaction. This helps reduce the impact of the autoimmune disease on the body.

Person who undergoes an organ transplant surgery must take immunosuppressant drugs as immune system of the person sees a transplanted organ as a foreign mass. As a result, immune system attacks the organ as it would attack any foreign cell. This can cause severe damage and lead to reject the transplanted organ. Immunosuppressant drugs weaken immune system to reduce body's reaction to the foreign organ. Thus, the drugs allow the transplanted organ to remain healthy and free from damage.

Important immunosuppressive agents include azathioprine, mycophenolatemofetil, cyclosporine, fingolimod, methotrexate, leflunomide, cyclophosphamide, chlorambucil, nitrogen mustard, tacrolimus and sirolimus.

CN1919184 discloses pharmaceutical formulations of an immunosuppressant dispersible tablets and process for preparation thereof.

EP0724581 relates to a pharmaceutical composition for preparing an aqueous intravenous formulation of an immunosuppressive mycophenolatemofetil.

CN101185623 relates to oral pharmaceutical composition in dry suspension dosage form comprises mycophenolatemofetil, filler, sweeteners, suspending agent and wetting agent.

U.S. Pat. No. 4,753,935 discloses oral suspension of an immunosuppressive agent mycophenolatemofetil. But this composition comprises the drug relatively low dose of 1 gram of active ingredient in 100 ml.

Currently available preparations of immunosuppressive agents are generally given orally in solid dosage forms of tablets or capsules, dry suspension or injections. The oral liquid dosage is more patient compliance as solid dosage form is not convenient for all patients to swallow. Further, immunosuppressive agents have strong bitterness that results a bitter taste and a feeling of numbness in the mouth. Injection dosage is also not convenient when the drug is needed to administer in high dose for more than once per day. Further, administration of injectable products either require hospitalization or medical staff.

Patients undergone organ transplant, they cannot take immunosuppressants in a solid dosage forms orally for 24-72 hr, so they are being treated with injectable immunosuppressants.

Hence, there is a need for development of oral liquid dosage form that is stable, having high bioavailability and palatability with patient compliance as well as can be administered with tube directly in to gastro-intestinal tract.

The present invention is directed to flavored liquid suspension composition of immunosuppressive agents that has masked bitter taste of active ingredient. Further, currently inventive formulation is exhibiting improved stability and palatability with high dose of active ingredient.

### OBJECTS OF THE INVENTION

The main object of the present invention is to provide oral pharmaceutical suspension of immunosuppressive agent with improved stability and palatability with high dose of active ingredient.

Another object of the present invention is to provide oral suspension of immunosuppressive agent with flavor that has masked bitter taste of the active ingredient and having patient compliance that eliminates difficulty of administration.

Another object of present invention is to provide oral suspension having dose accuracy, flexibility and uniformity of dosage.

Still another object of the present invention is to provide a convenient process for preparation of oral pharmaceutical suspension of immunosuppressive agent.

### SUMMARY OF THE INVENTION

The present invention relates to an oral pharmaceutical suspension of an immunosuppressive agent with improved stability, palatability with high dose of active ingredient. The present invention provides oral pharmaceutical suspension of an immunosuppressive agent with flavor that has masked unpleasant taste of the drug and patient compliance that eliminates difficulty of administration.

Another aspect of the present invention relates to oral suspension of an immunosuppressive agent that comprises an active ingredient with other pharmaceutically acceptable excipients such as vehicle, suspending agent, solvent or cosolvent, preservative, sweetener, antifoaming agent, wetting agent, buffering agent and flavouring agent. The present invention also provides a process for preparation thereof.

### DETAILED DESCRIPTION OF THE INVENTION

Immunosuppressant agents are anti-rejection drug used to prevent the body from rejecting a transplanted organ.

Immunosuppressant drugs greatly decrease the risks of rejection and protecting the new organ and preserving its function. These drugs act by blocking the immune system so that it is less likely to react against the transplanted organ. A wide variety of drugs are available to achieve this aim to reduce the risk of rejection.

The present invention relates to an oral pharmaceutical suspension of immunosuppressive agent with improved stability, palatability with high dose of active ingredient. The present invention comprises an active ingredient with other pharmaceutically acceptable excipients such as vehicle, suspending agent, solvent or cosolvent, preservative, sweetener, antifoaming agent, wetting agent, buffering agent and flavouring agent. The present invention also provides a convenient, easy process for preparation thereof.

The present invention provides oral pharmaceutical suspension of an immunosuppressive agent with flavor that has masked unpleasant taste of the drug with patient compliance that eliminates difficulty of administration.

In the present invention, the active pharmaceutical ingredient for oral suspension dosage form is selected from immunosuppressive agent such as azathioprine, mycophenolatemofetil, cyclosporine, fingolimod, methotrexate, leflunomide, cyclophosphamide, chlorambucil, nitrogen mustard, tacrolimus and sirolimus.

In a preferred embodiment of the present invention, the immunosuppressive agent is mycophenolatemofetil.

Mycophenolatemofetil is an immunosuppressant drug used to prevent rejection in organ transplantation. It is a prodrug of mycophenolic acid (MPA). Following oral administration, mycophenolatemofetil is rapidly absorbed and hydrolyzed to mycophenolic acid (MPA). It inhibits an enzyme needed for the growth of T cells and B cells. It reversibly inhibits inosine monophosphate dehydrogenase enzyme that controls the rate of synthesis of guanine monophosphate in the purine synthesis in the proliferation of B and T lymphocytes.

Mycophenolatemofetil is a broad-spectrum antibiotic and acting as antiviral, antifungal, antibacterial, anticancer, and antipsoriasis. USFDA approved the drug in May 1995 for use in kidney transplantation. Mycophenolatemofetil is marketed under the trade name CellCept.

Mycophenolatemofetil is having an empirical formula of C₂₃H₃₁NO₇ and a molecular weight of 433.4947, chemically 2-(morpholin-4-yl)ethyl (4E)-6-(4-hydroxy-6-methoxy-7-methyl-3-oxo-1,3-dihydro-2-benzofuran-5-yl)-4-methylhex-4-enoate, and having the chemical structure as follows:

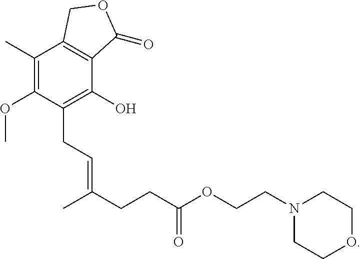 [SMILES: COc1c(C)c2c(c(O)c1C/C=C(\C)CCC(=O)OCCN1CCOCC1)C(=O)OC2]

One aspect of the present invention relates to oral pharmaceutical composition in suspension dosage comprises mycophenolatemofetil as active ingredient and pharmaceutically acceptable excipients. Pharmaceutically acceptable excipients may include vehicle, suspending agent, solvent or cosolvent, preservative, sweetener, antifoaming agent, wetting agent, buffering agent and flavouring agent. The present invention also provides a process for preparation thereof.

Vehicles used in the present pharmaceutical composition are mainly liquid which carry active ingredient and other excipients in dissolved or dispersed state. Pharmaceutical vehicles can be classified as aqueous vehicles and oily vehicles. Aqueous vehicles include water, hydro-alcoholic, polyhydric alcohols and buffers. Oily vehicles include vegetable oils, mineral oils, organic oily bases or emulsified bases. In the present invention, preferably water is used as vehicle.

Suspending agents help active pharmaceutical ingredients stay suspended in the formulation and prevent caking at the bottom of the container. A well-formulated suspension can be easily re-suspended by the use of moderate agitation or shaking. The main suspending agent employed in oral preparations can be selected from but not limited to carrageenan, colloidal silicone dioxide, cellulose ether, xanthan gum, sodium alginate, microcrystalline cellulose. In the present invention, preferably xanthan gum is used as suspending agent.

Solvents or cosolvents are water-miscible organic solvents used in liquid drug formulations to increase the solubility of poorly water soluble substances and enhance the chemical stability of a drug. Solvent or cosolvents can be selected from but not limited to water, ethanol, polyethylene glycols (PEG), sorbitol, glycerin, propylene glycol and benzyl alcohol. In the present invention, preferably glycerin is used as cosolvent to increase solubility of drug.

Preservatives are included in pharmaceutical dosage form and prevent the growth of microorganisms during the product manufacturing and shelf life. Preservatives can be selected from but not limited to benzoic acid, potassium sorbate, sodium benzoate, chlorobutanol, ethanol, butyl paraben, proply paraben and methyl paraben. In the present invention, preferably methyl paraben and proply paraben are used as preservatives.

Antifoaming agents are added in oral suspension pharmaceutical dosage form to lower the surface tension and cohesive binding of liquid phase. Antifoaming agents can be selected from but not limited to simethicone, organic phosphates, alcohols, paraffin oils, stearates and glycols. In the present invention, preferably simethicone emulsion is used as antifoaming agent.

Wetting agents are surfactants that lower the interfacial tension and contact angle of solid particles and liquid vehicle in suspensions. Wetting agents can be selected from but not limited to sodium lauryl sulphate, polysorbate 80, spans and lecithins. In the present invention, preferably polysorbate 80 is used as wetting agent as its lacks toxicity and have compatibility with most formulation ingredients.

Buffering agents provide stability and pH control to the pharmaceutical formulations. Buffering agents can be selected from but not limited to sodium acetate, sodium citrate, ammonium sulfate, sodium phosphate, disodium hydrogen phosphate, potassium citrate, citric acid monohydrate, trisodium citrate dihydrate. In the present invention, preferably sodium phosphate buffers are used as buffering agent.

Sweetening agents are added in liquid formulations that impart sweetness and improve patient compliance through taste masking. The main sweetening agents employed in oral preparations can be selected from but not limited to sucrose, liquid glucose, glycerol, sorbitol, saccharin sodium and aspartame. In the present invention, preferably sorbitol is used as sweetener.

Flavoring agents are added to increase patient acceptance of the drug by masking the specific taste sensations. Flavoring agent can be selected but not limited to essential oils including peppermint oil, orange oil, and lemon oil or can be selected from fruit flavors. In the present invention, preferably raspberry flavor is used.

Below table represents the composition of the present invention.

| Sr No | Name of Ingredients | Formula % w/w |
|---|---|---|
| 1 | API | 1-50% |
| 2 | Suspending agent | 1-10% |
| 3 | Cosolvent | 5-30% |
| 4 | Sweetener | 0.01-30% |
| 5 | Preservative | 0.001-1% |
| 6 | Antifoaming agent | 0.001-1% |
| 7 | Wetting agent | 0.001-10% |
| 8 | Buffering agent | 2-20% |
| 9 | Flavouring agent | 0.01-5% |
| 10 | Vehicle | Q.S. |

The oral pharmaceutical suspension of above composition is prepared by following steps but not limited to:
A) Take vehicle and add buffering agent and mix till it get dissolved;
B) Add and mix co-solvent until it get dispersed;
C) Add wetting agent and antifoaming agent one by one and mix till it gets dispersed or dissolved;
D) Add API and mix till it gets dispersed;
E) Add suspending agent, preservative, sweetener, flavouring agent one by one till it dissolved or dispersed;
F) Make volume up to desired batch size.

### EXAMPLES

The present invention can be described by way of examples only. They are not to be construed to limit the invention in any manner whatsoever. The following examples are intended to illustrate the various aspects of the invention, though without aiming to limit it.

Below table represents the composition of 1 gm/5 ml mycophenolatemofetil and excipients with its range are shown below:

### Example: Oral Suspension of Mycophenolatemofetil (1 gm/5 ml)

| Sr No | Name of Ingredients | Formula mg/ml |
|---|---|---|
| 1 | Mycophenolatemofetil | 200.0 |
| 2 | Xanthan gum | 2.0 |
| 3 | Glycerin | 100.0 |
| 4 | Sorbitol solution | 100.0 |
| 5 | Sodium methylparaben | 1.8 |
| 6 | Sodium propylparaben | 0.2 |
| 7 | Simethicone emulsion | 10.0 |
| 8 | Polysorbate 80 | 10.0 |
| 9 | NaH2PO4•2H2O | 1.7 |
| 10 | Na2HPO4•2H2O | 16.9 |
| 11 | Raspberry 502700 | 0.2 |
| 12 | Purified water | Q.S. to 1 ml |

The oral pharmaceutical suspension of above composition is prepared by following steps but not limited to:
A) Take vehicle and add buffering agents and mix till it get dissolved;
B) Add and mix co-solvent until it get dispersed;
C) Add wetting agent and antifoaming agent one by one and mix till it gets dispersed or dissolved;
D) Add API and mix till it gets dispersed;
E) Add suspending agent, preservative, sweetener, flavouring agent one by one till it dissolved or dispersed;
F) Make volume up to desired batch size.

1-6. (canceled)

7. A pharmaceutical oral suspension, comprising:
mycophenolate mofetil in an amount of about 200 mg/mL;
one or more cosolvents comprising ethanol, a polyethylene glycol, a sorbitol, glycerin, propylene glycol, benzyl alcohol, or a combination thereof in an amount of from about 5% w/w to about 30% w/w;
one or more pharmaceutically acceptable excipients selected from a suspending agent, a preservative, a sweetener, an antifoaming agent, a wetting agent, a buffering agent, and a flavoring agent; and
a vehicle comprising water.

8. The pharmaceutical oral suspension of claim 7, wherein the mycophenolate mofetil is present in an amount of 200 mg/mL.

9. The pharmaceutical oral suspension of claim 7, wherein the one or more cosolvents comprise ethanol, a sorbitol, glycerin, propylene glycol, or a combination thereof.

10. The pharmaceutical oral suspension of claim 7, wherein the one or more cosolvents comprise ethanol, a sorbitol, glycerin, propylene glycol, or a combination thereof in an amount of from about 5% w/w to about 20% w/w.

11. The pharmaceutical oral suspension of claim 7, wherein the one or more cosolvents comprise ethanol, a sorbitol, glycerin, propylene glycol, or a combination thereof in an amount of about 5% w/w to about 10% w/w.

12. The pharmaceutical oral suspension of claim 7, wherein the one or more cosolvents comprise glycerin in an amount of from about 5% w/w to about 20% w/w.

13. The pharmaceutical oral suspension of claim 7 comprising one or more suspending agents selected from a carrageenan, a colloidal silicon dioxide, a cellulose ether, a xanthan gum, a sodium alginate, and a microcrystalline cellulose.

14. The pharmaceutical oral suspension of claim 7 comprising one or more suspending agents selected from a carrageenan, a colloidal silicon dioxide, a cellulose ether, a xanthan gum, a sodium alginate, and a microcrystalline cellulose in an amount of from about 0.2% w/w % to about 10% w/w.

15. The pharmaceutical oral suspension of claim 7 comprising one or more preservatives selected from benzoic acid, potassium sorbate, sodium benzoate, chlorobutanol, butyl paraben or a pharmaceutically acceptable salt thereof, propyl paraben or a pharmaceutically acceptable salt thereof, and methyl paraben or a pharmaceutically acceptable salt thereof.

16. The pharmaceutical oral suspension of claim 7 comprising one or more preservatives selected from benzoic acid, potassium sorbate, sodium benzoate, chlorobutanol, butyl paraben or a pharmaceutically acceptable salt thereof, propyl paraben or a pharmaceutically acceptable salt thereof, and methyl paraben or a pharmaceutically acceptable salt thereof in an amount of from about 0.001% w/w to 1% w/w.

17. The pharmaceutical oral suspension of claim 7 comprising one or more preservatives selected from propyl paraben or a pharmaceutically acceptable salt thereof and methyl paraben or a pharmaceutically acceptable salt thereof in an amount of from about 0.2% w/w to about 1% w/w.

18. The pharmaceutical oral suspension of claim 7 comprising one or more antifoaming agents selected from a simethicone, an organic phosphate, a paraffin oil, a stearate, and a glycol.

19. The pharmaceutical oral suspension of claim 7 comprising an antifoaming agent comprising a simethicone in an amount of from 0.001% w/w to about 1% w/w.

20. The pharmaceutical oral suspension of claim 7 comprising one or more wetting agents selected from sodium lauryl sulfate, a polysorbate, a sorbitan fatty acid ester, and a lecithin.

21. The pharmaceutical oral suspension of claim 7 comprising one or more wetting agents selected from sodium lauryl sulfate, a polysorbate, a sorbitan fatty acid ester, and a lecithin in an amount of from about 0.001% w/w to about 10% w/w.

22. The pharmaceutical oral suspension of claim 7 comprising one or more wetting agents selected from sodium lauryl sulfate, a polysorbate, a sorbitan fatty acid ester, and a lecithin in an amount of from about 0.001% w/w to about 1% w/w.

23. The pharmaceutical oral suspension of claim 7 comprising polysorbate 80 in an amount of from about 0.001% w/w to about 1% w/w.

24. The pharmaceutical oral suspension of claim 7 comprising one or more buffering agents selected from citric acid, potassium citrate, sodium citrate, trisodium citrate, sodium acetate, ammonium sulfate, sodium dihydrogen phosphate, and disodium hydrogen phosphate.

25. The pharmaceutical oral suspension of claim 7 comprising one or more buffering agents selected from citric acid, potassium citrate, sodium citrate, trisodium citrate, sodium acetate, ammonium sulfate, sodium dihydrogen phosphate, and disodium hydrogen phosphate in an amount of from about 2% w/w to about 20% w/w.

26. An immunosuppressive method, which comprise administering a therapeutically effective amount of the pharmaceutical oral suspension of claim 7 to a patient in need thereof.
